(19) United States
(12) Patent Application Publication
(10) Pub. No.: US 2013/0098370 A1
(43) Pub. Date: Apr. 25, 2013

(54) Partial weight bearing device

(71) Applicant: Clay Thomas Cacas, Terrell, TX (US)

(72) Inventor: Clay Thomas Cacas, Terrell, TX (US)

(73) Assignee: Clay Thomas Cacas, Terrell, TX (US)

(21) Appl. No.: 13/317,603

(22) Filed: Oct. 25, 2011

(51) Int. Cl. A61F 13/00

(52) U.S. Cl. 128/845

(57) ABSTRACT

A lightweight easy to use device for the partial weight bearing during sitting or other activities the results in pressure being applied to areas of the body for extended periods of time. The device is made by attaching material that does not slide when wrapped around a person and is in contact with said person's skin, to a device that creates a force to partially support the weight of the person using the device and remove the pressure from other weight bearing areas.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF INVENTION

1. Field of Invention

This invention relates generally to comfort devices and more particularly to a living aid often used to provide comfort and relieve pressure from certain parts of the body and to help with the healing and prevention of pressure wounds.

2. Prior Art

There exist many types of devices and cushions that can help provide some comfort for persons that sit or lay down for long periods of time. All of these devices are designed to decrease the pressure on the coccyx by having the cushion support the person in other areas. The full weight is still being supported from pressure points below the coccyx. The pressure caused by this weight can cause discomfort and ulcers, often called pressure wounds. These wounds are very painful and can be fatal if the wound does not heal.

While these devices do provide some comfort, there exists a need for a apparatus having new features whereby the device provides comfort and partially supports the persons weight from a point above the coccyx. An object of the present invention is to provide an improved partial weight bearing device to meet these needs.

### BRIEF SUMMARY OF THE INVENTION

This Invention is an apparatus that provides partial weight support of a person from points above the coccyx for the purpose of decreasing the pressure placed on the coccyx to allow more blood flow.

The invention is a partial weight bearing device that consists of a semi-rigid frame that is shaped in a way that when it is attached to a person a force up is created above the coccyx as the person sits down and the device is compressed causing the recoil to create a force up. Attached to the semi-rigid frame is cloth material with a gripping material attached to the cloth. The gripping material goes around the person's skin making a harness that does not slip when worn by a person using this device.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

It is therefore an object of the present invention to provide a new and improved device that will have all of the advantages of prior art devices and none of the disadvantages.

A better understanding of the objects, advantages, feature, properties and relationships of the invention will be obtained from the following detailed description and accompanying drawings which set forth an illustrative embodiment and is indicative of the various ways in which the principles of the invention may be employed.

### BRIEF DESCRIPTION OF THE DRAWINGS

FIG. 1 front view of present invention shown with preferred embodiment;

FIG. 2 side view of a metal frame shown with preferred embodiment;

### DETAILED DESCRIPTION

As such, those skilled in those skilled in the art will appreciate that the conception, upon which this disclosure is based, may readily be utilized as a basis for the beginning of other structures, methods and systems for carrying out the several purposes of the present invention. It is important, therefore, that the claims be regarded as including such equivalent constructions insofar as they do not depart from the spirit and scope of the present invention.

While the inventions contained herein can be used in all types of pressure reducing devices it will be described hereinafter in the context of a partial weight bearing device as the preferred embodiment thereof.

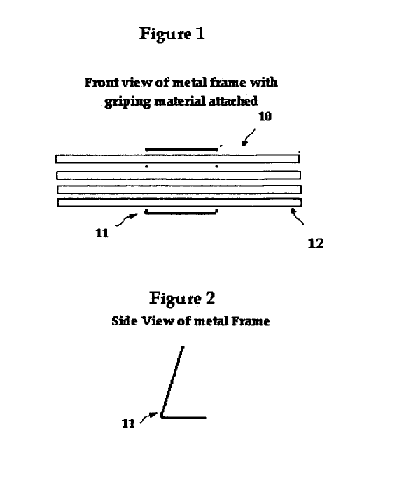

With reference now to the drawings, and in particular FIG. 1 thereof, a partial weight bearing device (10) will be described.

The present invention (10) consist of a metal frame (11) covered with a cloth with several griping strips (12) attached to it. The metal frame shown in FIG. 2 (11) is constructed in a shape that will create a force up from the recoil of the frame (11) when the person wearing the device sits down. The gripping strips (12) prevent the device from slipping when the recoil force from created from the frame (11) is applied. The force up is transferred to the gripping strips (12) to create a partial weight bearing support at a point above the coccyx. The cloth material with the gripping strips could have many shapes or materials. The vest like device could also be supported by many devices as long as the force applied to the vest results in a force up being generated from a point above the wound area.

What is claimed is:

1. A unitary support device for supporting a body part with an pressure wound or a part that is susceptible to developing a pressure wound comprising: a gripping material attached to a device designed to decrease the pressure on the body part by supporting the body part from a pressure point other than the point under the wound or a point where wounds are known to occur.

2. The support device according to claim 1, where the device design to partially support the body part is a semi rigid frame that allows the gripping material to be attached to it, and the supporting device has a base that creates a recoil force when the frame is compressed.

3. The support device according to claim 1, where the device design to partially support the body part is equipped with a Velcro support system that provides the force to the support device.

4. The support device according to claim 1, where the device design to partially support the body part is equipped with areas that allow for the attachment of support devices that originate from points above the support device, to provide a force to the support device.
